(19) United States
(12) Patent Application Publication
(10) Pub. No.: US 2026/0207592 A1
(43) Pub. Date: Jul. 23, 2026

(54) PHARMACEUTICAL SUSPENSION OF NILOTINIB

(71) Applicant: RENATA PHARMACEUTICALS (IRELAND) LIMITED, Dublin (IE)

(72) Inventors: Syed S. Kaiser KABIR, Dhaka (BD); Syed Omar KABIR, Dhaka (BD); Ganesh Vinayak GAT, Dhaka (BD); Boorugu RAMBABU, Dhaka (BD); A.H.M. Masbahur RAHMAN, Dhaka (BD)

(21) Appl. No.: 19/144,004

(22) PCT Filed: Dec. 29, 2023
(86) PCT No.: PCT/IB2023/063367

§ 371 Date: Jun. 27, 2025

Related U.S. Application Data
(60) Provisional application No. 63/477,787, filed on Dec. 29, 2022.

(51) Int. Cl. A61K 31/506; A61K 9/10; A61K 47/02; A61K 47/10; A61K 47/12; A61K 47/14; A61K 47/20; A61K 47/26; A61K 47/34; A61K 47/36; A61K 47/38

(52) U.S. Cl. CPC A61K 31/506; A61K 9/10; A61K 47/02; A61K 47/10; A61K 47/12; A61K 47/14; A61K 47/20; A61K 47/26; A61K 47/34; A61K 47/36; A61K 47/38

(57) ABSTRACT

A pharmaceutical oral suspension comprising: Nilotinib or a pharmaceutically acceptable salt thereof Water; a suspending agent; a stabilizing agent; a buffering agent and one or more pharmaceutically acceptable excipients.

### FIELD OF THE INVENTION

The present invention relates to a field of an oral liquid suspension in general, and in particular to an oral liquid pharmaceutical suspension of nilotinib or pharmaceutically acceptable salts thereof and a process for preparing the same.

### BACKGROUND OF THE INVENTION

Nilotinib is 4-Methyl-3-[[4-(3-pyridinyl)-2-pyrimidinyl]amino]-N-[5-(4-methyl-1H-imidazol-1-yl)-3-(trifluoromethyl)phenyl] benzamide. A particularly useful salt of nilotinib is nilotinib hydrochloride monohydrate. These therapeutic compounds have utility as inhibitors of the protein tyrosine kinase (TK) activity of Bcr-Abl. Examples of conditions that may be treated by such therapeutic compounds include, but are not limited to, chronic myeloid leukemia and gastrointestinal stromal tumors. Tasigna™ oral capsule (nilotinib) has been developed for chronic myeloid leukemia and gastrointestinal stromal tumors. Nilotinib is commercially available as 50 mg oral capsule, 100 mg oral capsule, 150 mg oral capsule and 200 mg oral capsule. Nilotinib is very slightly soluble drug in water and slightly soluble drug in alcohol, belonging to the BCS class IV, and its solubility in water is 0.2 μg/ml. In order to ensure bioavailability, the Nilotinib capsules developed by Novartis use micronized Nilotinib along with Poloxamer 188 as a solubilizer to increase the solubility of the drug. Novartis further discloses, problem of nilotinib overdose in combination with alcohol.

It is important that pharmaceutical products be safe and effective. Even if a pharmaceutical product is stable immediately after production, it may be easily decomposed or denatured on storage or during distribution. Such a drug is not safe and effective as a pharmaceutical product. Therefore, the stability of the drug is extremely important for pharmaceutical products.

However, in the case of oral solid formulations of Nolitonib there has been a defect that it is difficult for young children and old patients to take them. Since the formulation is to be administered by dispersing in apple sauce, it leads to spillage, dose inaccuracy and patient noncompliance. Another problem is that since very slightly soluble drugs dissolve only in a low concentration, thus a single dose of the suspension becomes too large. Moreover, Tasigna™ label suggested complex process such as, to open capsule and sprinkle on applesauce with patients having difficulty in swallowing and time limit to use said mixture is 15 minutes, which complex process and needs to repeat every time of dosing. This adds to the non-compliance, inconsistent dose administration and caregivers are exposed to the cytotoxic drug. A liquid dosage form is preferable for pediatrics and geriatrics but it has stability issues. Further formulating a suspension dosage form with a high dose and low solubility drug is difficult. It is desirable to have a liquid dosage form for pediatric and geriatric patients with improve dosage accuracy, patient compliance with a stable and palatable formulation.

An improved oral liquid formulation of Nilotinib was tried.
U.S. Pat. No. 7,169,791 discloses compound nilotinib or its pharmaceutically acceptable salts thereof.
U.S. Pat. No. 8,163,904 discloses the mono hydrochloride salt of Nilotinib.
U.S. Pat. Nos. 8,501,760 and 8,293,756 discloses solid oral pharmaceutical composition, especially capsules and granules, of Nilotinib or a salt thereof and excipients.
WO2019/162756 discloses a liquid composition of an anticancer drug and one or more pharmaceutically acceptable excipients. The liquid composition is selected from the group comprising of liquids, liquid dispersions, suspensions, solutions, emulsions, sprays, spot-on, syrups, elixirs or concentrates.
PCT publication WO2016087952 discloses gastro-retentive extended release suspension. PCT publication WO2020039264 discloses pharmaceutical oral suspension formulation comprising anticancer API selected from ibrutinib, nilotinib, dasatinib, sunitinib, sorafenib, erlotinib, and capecitabine and sugar alcohol. It further discloses sugar alcohol selected from polyhydric alcohols, polyols and hydrogenated sugars. Nilotinib suspension prepared as per PCT publication WO2020039264, shows lumps formation, leads to storage, handling, dispensing and dosing related problems.

It has been surprisingly found that a stable suspension dosage form of Nilotinib or pharmaceutically acceptable salts thereof can be prepared which overcomes the problems of low solubility, low bioavailability and large dose. Since the concentration of drug is high, it leads to phase separation and stability issues like amide degradation. Nilotinib or pharmaceutically acceptable salts thereof is prone to amide hydrolysis in acidic condition and the Nilotinib API has a better solubility in acidic condition. Use of Sodium Chloride in the suspension formulation increases ionic strength, modify the viscosity of the suspending agent, hence increases solubility of a suspension which has high solid content, avoids phase separation, sedimentation and leads to a stable suspension formulation of Nilotinib or pharmaceutically acceptable salts thereof. The said composition is cost effective and process of obtaining it is less complex. The present inventors have also surprisingly found that an aqueous oral Nilotinib or pharmaceutically acceptable salts thereof can be stored at room temperature and inhibit the formation of the impurity, while maintaining a functioning suspension.

### SUMMARY OF THE INVENTION

In accordance with the principal aspect of the present invention, there is provided an oral pharmaceutical suspension comprising nilotinib or pharmaceutically acceptable salts thereof that exhibits stable aqueous oral suspension.

One aspect of the invention is an aqueous oral suspension comprising (i) Nilotinib or pharmaceutically acceptable salts thereof, (ii) suspending agent, (iii) buffering agent, (iv) viscosity modifier and (v) surfactant and (vi) optionally one or more pharmaceutically acceptable excipients, where the suspension is stored at ambient temperature (e.g., 25° C.). The suspension is formulated such that it remains stable during storage and the nilotinib or pharmaceutically acceptable salts thereof does not precipitate out.

Yet another aspect is a method of dispensing to a patient an aqueous oral suspension comprising (i) nilotinib or pharmaceutically acceptable salts thereof, (ii) suspending agent, (iii) stabilizing agent (iv) surfactant, and (v) optionally one or more pharmaceutically acceptable excipients.

In some embodiments, the suspending agent is selected from gelatin, crosslinked polyacrylic acid, polymethacrylic acid, polyhydroxyethyl methacrylic acid, hydroxypropyl methyl cellulose, polyethylene glycol, sodium carboxymethyl cellulose, hyaluronic acid, chitosan, polycarbophil, pectin, copolymers of dextran, polyacrylamide, acacia, copolymer of caprolactone and ethylene oxide, carbopol 934, tragacanth, eudragit, polyvinyl pyrrolidone, polyacrylate and polyacrylate copolymer resins, celluloses and cellulose derivatives for example methyl-, ethyl- and propyl celluloses; micro crystalline cellulose, hydroxyalkyl-celluloses, hydroxyl propyl celluloses, hydroxylpropylalkyl celluloses, hydroxyl ethyl cellulose, co-processed microcrystalline cellulose and sodium carboxymethyl cellulose and the like including xanthan gum, polyvinyl resins, polyethylene glycol, polyethylene oxide, sorbitol, sucrose, xylitol, dextrose, fructose, maltitol, sugar, sodium alginate, or a combination thereof.

In another aspect, the present invention relates to an aqueous oral suspension comprising nilotinib or a pharmaceutically acceptable salts thereof and one or more viscosity modifier, wherein the suspension is free or substantially free (e.g. contains less than about 10% v/v, such as less than about 5% v/v, less than about 2.5% v/v, less than about 2% v/v, less than about 1.5% v/v, less than about 1% v/v, less than about 0.5% w/v, less than about 0.3% v/v, less than about 0.2% v/v, less than about 0.1% v/v, less than about 0.05% v/v, or less than about 0.01% v/v) of alcohol.

Yet another aspect is a method of dispensing to a patient an aqueous oral suspension comprising (i) nilotinib or pharmaceutically acceptable salts thereof, (ii) stabilizing agent, (iii) optionally one or more polyol.

In other aspect, the suspension composition comprises about 5% (w/v) to about 50% (w/v) of polyol (also known as sugar alcohol), or about 15% (w/v) to about 50% (w/v) of polyol. Non limiting example of polyols that can be used in the inventive compositions include sorbitol, xylitol, maltitol, glycerine, propylene glycol, erythritol and combinations thereof. In other embodiments, the liquid composition comprises about 15% (w/v) to about 40% (w/v) sorbitol or 15% (w/v) to about 25% (w/v) sorbitol, or about 16% (w/v) to about 26% (w/v) sorbitol or about 18% (w/v) to about 24% (w/v) sorbitol and in other embodiments, the liquid composition comprises about 21% (w/v) sorbitol. In other embodiments the liquid composition comprises about 15% (w/v) to about 40% (w/v) of a combination of sorbitol and maltitol. In other embodiments, the liquid composition comprises about 15% (w/v) to 25% (w/v) of maltitol and in other embodiments, the composition comprises about 20% (w/v) maltitol. In other embodiments the liquid composition comprises about 1% (w/v) to about 25% (w/v) of glycerine, and in other embodiments the liquid composition comprises about 5% (w/v) glycerine. In other embodiments the liquid composition comprises about 5% to about 50% propylene glycol (w/v).

In certain aspects, the co-solvents are organic solvents such as propylene glycol, polyethylene glycol, and combinations thereof that are pharmaceutically acceptable based on the desired formulation.

In certain aspects, the formulation contains from about 5% to about 50% by weight polyethylene glycol.

In one aspect of any of the aqueous oral suspension described herein, the suspension may comprises a sweetener, such as sucralose, sucrose, or sodium saccharin. In one embodiment, the suspension comprises from about 0 to about 50% w/v sweetener. In one embodiment, the suspension comprises from about 0.001 to about 1% w/v sucralose, such as about 0.025% w/v sucralose. In other embodiments, the concentration of sucrose is about 5% (w/v) to about 25% (w/v) or 2% (w/v) to about 30% (w/v) and in other embodiments, the concentration of sucrose is about 15% (w/v).

In other aspects, the sweetener of the liquid composition is an artificial sweetener (also known in the art as a “high intensity sweetener”). Exemplary artificial sweeteners include sucralose, acesulfame potassium, aspartame, and the saccharins. In other embodiments, the artificial sweetener is selected from sucralose and saccharin. In other embodiments, the sucralose is present at a concentration of about 5% (w/v); in other embodiments, the concentration of sucralose is from about 10% (w/v) to about 15% (w/v). In other embodiments, the concentration of saccharin is about 5% (w/v) to about 25% (w/v) or 2% (w/v) to about 30% (w/v) and in other embodiments, the concentration of saccharin is about 15% (w/v).

In one aspect of any of the aqueous oral suspension described herein, the suspension comprises from about 1 to about 50% w/v of nilotinib or pharmaceutically acceptable salts thereof, such as from about 10 to about 40% w/v of nilotinib or pharmaceutically acceptable salts thereof or from 15 to about 40% w/v of nilotinib or pharmaceutically acceptable salts thereof.

In one aspect of any of the aqueous oral suspension described herein, the suspension comprises about 20% w/v of Nilotinib or pharmaceutically acceptable salts thereof, In another embodiment, the suspension comprises about 30% w/v of Nilotinib or pharmaceutically acceptable salts thereof.

In one aspect of any of the aqueous oral suspension described herein are one or more acids. Suitable acids include but are not limited to acetic acid, ascorbic acid, citric acid, fumaric acid, lauric acid, glacial, sorbic acid, malic acid, succinic acid, tartaric acid, phosphoric acid, hydrochloric acid, amino acid and mixtures thereof. Organic acids, which enhance the taste of the suspension, are especially useful. Citric acid, for example, adds tartness that is a taste enhancer, and may play a role in overall stability of the suspension. Suitable amino acid include but are not limited to lysine, alanine, glutamic acid, tryptophan and mixture thereof. The concentration of the acid in the solvent system is in the range of about 0.1% to about 10.0% or about 0.25% to about 5.0% or about 0.5% to about 3% or about 2%.

In one aspect of any of the pharmaceutical suspension described herein, the one or more preservatives comprise Bronopol, Imidurea, Potassium Sorbate, Phenoxyethanol, Phenylmercuric Acetate, Butylparaben, Benzyl Alcohol, Phenylmercuric Borate, Chlorocresol, Benzethonium Chloride, Phenylethyl Alcohol, Benzalkonium Chloride, Methylparaben, Hexetidine, Chlorobutanol, Ethylparaben, Propylparaben, Sodium Benzoate, Potassium Benzoate, Sorbic Acid, Cresol, Propylparaben Sodium, Cetylpyridinium Chloride, Phenylmercuric Nitrate, Chloroxylenol, Propionic Acid, Phenol, Thimerosal, Sulfur Dioxide, Boric Acid, Edetic Acid, Sodium Propionate, Calcium Chloride, Sodium Acetate, Sodium Sulfite, Benzoic Acid, Monothioglycerol, Cetrimide, Calcium Acetate, Butylene Glycol, Sodium Metabisulfite, Alcohol, Propyl Gallate, Potassium Metabisulfite, Sodium Lactate, Chlorhexidine, Calcium Lactate, Pentetic Acid, Glycerin, Propylene Glycol Alginate, Sodium Borate, Magnesium Trisilicate, Isopropyl Alcohol, Dimethyl Ether, Propylene Glycol, Butylated Hydroxyanisole, Pyrrolidone, Lactic Acid, Sodium Lauryl Sulfate and Dimethyl Sulfoxide or a combination thereof. In another embodiment of any of the aqueous oral suspension described herein, the one or more preservatives comprise methylparaben, propylparaben, or a combination thereof. For instance, the oral suspension may comprise methylparaben and propylparaben.

For instance, the oral pharmaceutical suspension may comprise methylparaben and propylparaben. The suspension includes an effective amount of preservatives (such as methylparaben, propylparaben, sodium benzoate, sodium methylparaben, or a combination thereof) to inhibit microbial contamination of the suspension. In one embodiment, the suspension includes from about 0 to about 4% (such as from about 0 to about 1% or from about 0 to about 0.5%) of preservatives, such as methylparaben, propylparaben, or a combination thereof. In another embodiment, the suspension includes from about 0 to about 0.2% of preservatives, such as methylparaben, propylparaben, or a combination thereof.

In one aspect of any of the aqueous pharmaceutical suspension described herein, the suspension comprises one or more flavoring agents, such as, e.g., natural grape flavor. Other suitable flavoring agents include but, are not limited to, cotton candy, raspberry, strawberry, lemon-lime, apple, mint, licorice, orange, lemon, grapefruit, caramel, vanilla, cherry, grape flavors, tutti-frutti, cherry flavors, combinations thereof; and the like. In one embodiment, the suspension comprises from about 0.01 to about 2% w/v flavoring agent (e.g., natural grape flavor), such as about 1.0% w/v flavoring agent.

In some aspects, the stable oral pharmaceutical suspension of the present application comprises of at least one surfactants selected from the group of nonionic, anionic, cationic and zwitterionic surfactants or mixtures thereof.

Suitable non-limiting examples of the surfactants used in the compositions of the present invention includes, but not limited to, polyethoxylated fatty acids like esters of lauric acid, oleic acid, and stearic acid, PEG-fatty acid diesters like PEG-20 dilaurate, PEG-fatty acid mono- and di-ester mixtures, alcohol-oil transesterification products like PEG-35 castor oil, Polyoxy 35 castor oil, polyoxyl 40 hydrogenated castor oil, etc., polyglycerized fatty acids like polyglyceryl oleate, etc., propylene glycol fatty acid esters like propylene glycol monolaurate etc., mixtures of propylene glycol esters-glycerol esters like oleic acid esters of propylene glycol and glycerol, etc., sterol and sterol derivatives like PEG-24 cholesterol ether etc., polyethylene glycol sorbitan fatty acid esters like PEG-20 sorbitan monolaurate etc., polyethylene glycol alkyl ethers like PEG-3 oleyl ether, etc., sugar esters like sucrose monopalmitate etc., polyethylene glycol alkyl phenols like Octoxynol-1 etc., sorbitan fatty acid esters like sorbitan monolaurate, lower alcohol fatty acid esters like ethyl oleate, etc., anionic surfactants include fatty acid salts and bile salts. Additional exemplary surfactants include, but are not limited to: polyoxyethylene alkylethers; polyethylene glycol fatty acid esters; polyethylene glycol glycerol fatty acid esters; polyoxyethylene sorbitan fatty acid esters; polyglycerol fatty acid esters; polyoxyethylene glycerides; polyoxyethylene vegetable oils; polyoxyethylene hydrogenated vegetable oils, alkylglucosides; alkylmaltosides; alkylthioglucosides; lauryl macrogolglycerides; Ionic surfactants include sodium oleate, sodium lauryl sulfate, sodium lauryl sarcosinate, sodium dioctyl sulfosuccinate, sodium cholate, and sodium taurocholate; Gelucire® 44/14, etc.

The at least one surfactant be present in the stable oral liquid pharmaceutical suspension disclosed herein in a variety of concentrations. For example, the stable oral liquid pharmaceutical suspension disclosed herein can comprise at least one surfactant of at least 0.25% by weight, at least 2% by weight, at least 5% by weight, at least 10% by weight, at least 15% by weight, at least 30% by weight, or a percentage between any two of the above values, based on the total weight of the composition. In some embodiments, the nilotinib composition of present application comprises of at least one surfactant in an amount of from about 0.25% to about 20% by weight, or from about 2% to about 15% by weight, or from about 2% to about 10% by weight, or from about 3% to about 10% by weight, based on the total weight of the composition.

In one aspect of any of the aqueous oral suspension described herein, the pH of the aqueous oral suspension ranges from about 2 to about 7. In another of any of the aqueous oral suspension described herein, the pH of the aqueous oral suspension ranges from about 2.0 to about 4. For instance, the pH may be 2.7, 2.8, 2.9, 3, 3.1, 3.2, 3.3, 3.4, or 3.5.

In some aspects, the stable oral liquid pharmaceutical suspension of the present application may comprises of at least one buffering agents. Non-limiting examples of buffering agents include, but are not limited to acetate, amino acids, ammonium sulfate, benzoate, bicarbonate, borate, citrate, citric acid monohydrate, disodium hydrogen phosphate, glutamate, lactate, meglumine, potassium citrate, sodium acetate, sodium citrate, sodium phosphate, sulfate, tartrate, triethanolamine, TRIS, trisodium citrate dehydrate, and any combination thereof.

In one aspect of any of the aqueous oral suspension described herein, the suspension may comprises stabilizing agents. Suitable stabilizing agents which may be incorporated into composition of the present invention include, e.g., monovalent or multivalent metal cations. Non-limiting examples salts are inorganic salts, include calcium sulfate, sodium chloride, potassium sulfate, sodium carbonate, lithium chloride, tripotassium phosphate, sodium borate, potassium bromide, potassium fluoride, sodium bicarbonate, calcium chloride, magnesium chloride, sodium citrate, sodium acetate, calcium lactate, magnesium sulfate and sodium fluoride or any of the commonly employed salts.

For the purpose of this invention, stabilizing agent and viscosity modifier are the same.

In another aspect stabilizing agent may comprises multivalent metal cations. In another embodiments, the stabilizing agents are bivalent. In yet another embodiments, bivalent cation salts is sodium chloride.

In one aspect of any of the aqueous oral suspension described herein, the suspension comprises from about 0.1 to about 10% w/v of stabilizing agent, such as from about 0.2 to about 5% w/v of stabilizing agent.

In one aspect of any of the aqueous oral suspension described herein, the suspension comprises from about 0.1 to about 10% w/v of viscosity modifier, such as from about 0.2 to about 5% w/v of viscosity modifier.

In one aspect of any of the aqueous oral suspension described herein, the suspension may comprises anti-oxidant. Non-limiting examples of anti-oxidant included, but are not limited to propyl gallate, lecithin, Vitamin E tocopherol, sesamin, sesamol, sesamolin, alpha tocopherol, ascorbic acid, ascorbyl palmitate, fumaric acid, citric acid, malic acid, and sodium metabisulphite, disodium EDTA, and combinations of any of the foregoing.

Another aspect of the present invention is to provide an aqueous oral suspension composition containing, while having a reduced bitter taste of the drug.

A further aspect of the present invention is to provide a suspension composition which is easy to take.

The details of one or more aspect of the inventions are set forth in the description below.

### BRIEF DESCRIPTION OF THE DRAWINGS

FIG. 1: Shows a comparison of dissolution data with graph with various excipient, Dissolution in pH 4.5 Acetate buffer+0.5% SLS, Apparatus-II, RPM-50, Volume 900 mL.

FIG. 2: Shows a comparison of dissolution data with graph with various excipient, Dissolution in pH 6.8 Phosphate Buffer+0.15% SLS, Apparatus-II, RPM-50, volume 900 mL

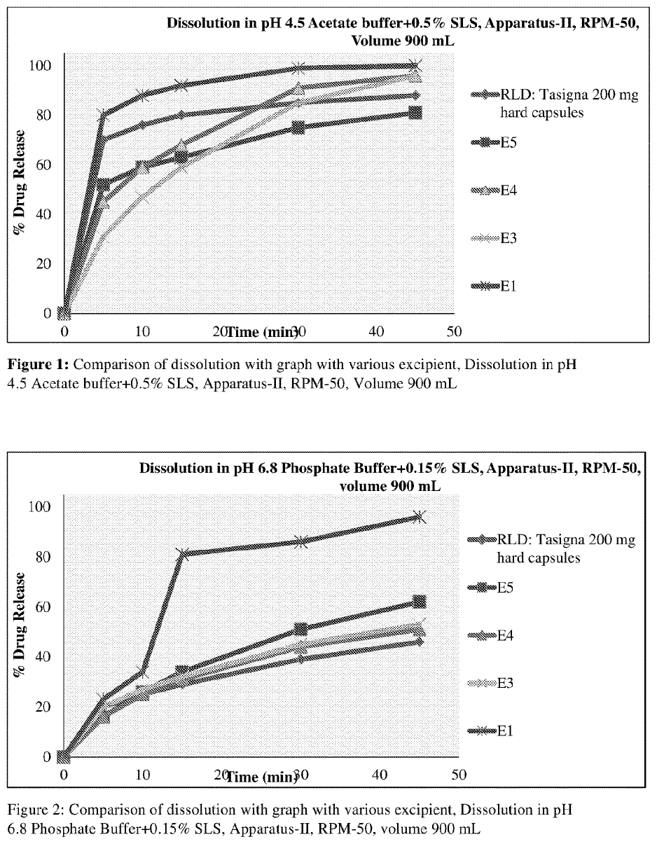

### BRIEF DESCRIPTION

Before the present compositions and methods are described, it is to be understood that this invention is not limited to particular compounds, formulas or steps described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a. compound” includes a plurality of such compounds and reference to “the step” includes reference to one or more step and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

In accordance with the principal aspect of the present invention, there is provided oral pharmaceutical suspension comprising nilotinib or pharmaceutically acceptable salts thereof that exhibits stable aqueous oral suspension.

One aspect of the invention is an aqueous oral suspension comprising (i) Nilotinib or pharmaceutically acceptable salts thereof (ii) suspending agent, (iii) buffering agent, (iv) viscosity modifier (v) surfactant and (vi) optionally one or more excipient, where the suspension is stored at ambient temperature (e.g., 25° C.). The suspension is formulated such that it remains stable during storage and the nilotinib or pharmaceutically acceptable salts thereof does not precipitate out.

Yet another aspect is a method of dispensing to a patient an aqueous oral suspension comprising (i) nilotinib or pharmaceutically acceptable salts thereof, (ii) suspending agent, (iii) stabilizing agent and (iv) optionally one or more excipient.

In some embodiments, the suspending agent is selected from gelatin, crosslinked polyacrylic acid, polymethacrylic acid, polyhydroxyethyl methacrylic acid, hydroxypropyl methyl cellulose, polyethylene glycol, micro crystalline cellulose, hydroxyl ethyl cellulose, sodium carboxymethyl cellulose, hyaluronic acid, chitosan, polycarbophil, pectin, copolymers of dextran, polyacrylamide, acacia, copolymer of caprolactone and ethylene oxide, carbopol 934, tragacanth, eudragit. polyvinyl pyrrolidone, polyacrylate and polyacrylate copolymer resins, celluloses and cellulose derivatives for example methyl-, ethyl- and propyl celluloses; hydroxyalkyl-celluloses, hydroxyl propyl celluloses, hydroxylpropylalkyl celluloses, co-processed microcrystalline cellulose and sodium carboxymethyl cellulose and the like including xanthan gum, polyvinyl resins, polyethylene oxide, sorbitol, sucrose, xylitol, dextrose, fructose, maltitol, sugar, sodium alginate, or a combination thereof.

In another aspect, the present invention relates to an aqueous oral suspension comprising nilotinib or pharmaceutically acceptable salts thereof and one or more viscosity modifier, wherein the suspension is free or substantially free (e.g. contains less than about 10% v/v, such as less than about 5% v/v, less than about 2.5% v/v, less than about 2% v/v, less than about 1.5% v/v, less than about 1% v/v, less than about 0.5% w/v, less than about 0.3% v/v, less than about 0.2% v/v, less than about 0.1% v/v, less than about 0.05% v/v, or less than about 0.01% v/v) of alcohol.

Yet another aspect is a method of dispensing to a patient an aqueous oral suspension comprising (i) nilotinib or pharmaceutically acceptable salts thereof, (ii) stabilizing agent, (iii) optionally one or more polyol.

In other aspect, the suspension composition comprises about 5% (w/v) to about 50% (w/v) of polyol (also known as sugar alcohol), or about 15% (w/v) to about 50% (w/v) of polyol. Non limiting example of polyols that can be used in the inventive compositions include sorbitol, xylitol, maltitol, glycerine, propylene glycol, erythritol and combinations thereof. In other embodiments, the liquid composition comprises about 15% (w/v) to about 40% (w/v) sorbitol or 15% (w/v) to about 25% (w/v) sorbitol, or about 16% (w/v) to about 26% (w/v) sorbitol or about 18% (w/v) to about 24% (w/v) sorbitol and in other embodiments, the liquid composition comprises about 21% (w/v) sorbitol. In other embodiments the liquid composition comprises about 15% (w/v) to about 40% (w/v) of a combination of sorbitol and maltitol. In other embodiments, the liquid composition comprises about 15% (w/v) to 25% (w/v) of maltitol and in other embodiments, the composition comprises about 20% (w/v) maltitol. In other embodiments the liquid composition comprises about 1% (w/v) to about 25% (w/v) of glycerine, and in other embodiments the liquid composition comprises about 5% (w/v) glycerine. In other embodiments the liquid composition comprises about 5% to about 50% polyethylene glycol (w/v).

In certain aspects, the co-solvents are organic solvents such as propylene glycol, polyethylene glycol, and combinations thereof that are pharmaceutically acceptable based on the desired formulation.

In certain aspects, the formulation contains from about 5% to about 50% by weight polyethylene glycol.

In one aspect of any of the aqueous oral suspension described herein, the suspension may comprise a sweetener, such as sucralose, sucrose, or sodium saccharin. In one embodiment, the suspension comprises from about 0 to about 50% w/v sweetener. In one embodiment, the suspension comprises from about 0.001 to about 1% w/v sucralose, such as about 0.025% w/v sucralose. In other embodiments, the concentration of sucrose is about 5% (w/v) to about 25% (w/v) or 2% (w/v) to about 30% (w/v) and in other embodiments, the concentration of sucrose is about 15% (w/v).

In other aspects, the sweetener of the liquid composition is an artificial sweetener (also known in the art as a “high intensity sweetener”). Exemplary artificial sweeteners include sucralose, acesulfame potassium, aspartame, and the saccharins. In other embodiments, the artificial sweetener is selected from sucralose and saccharin. In other embodiments, the sucralose is present at a concentration of about 5% (w/v); in other embodiments, the concentration of sucralose is from about 10% (w/v) to about 15% (w/v). In other embodiments, the concentration of saccharin is about 5% (w/v) to about 25% (w/v) or 2% (w/v) to about 30% (w/v) and in other embodiments, the concentration of saccharin is about 15% (w/v).

In one aspect of any of the aqueous oral suspension described herein, the suspension comprises from about 1 to about 50% w/v of nilotinib or pharmaceutically acceptable salts thereof, such as from about 10 to about 40% w/v of nilotinib or pharmaceutically acceptable salts thereof, or from 15 to about 40% w/v of nilotinib or pharmaceutically acceptable salts thereof. In one aspect of any of the aqueous oral suspension described herein, the suspension comprises about 20% w/v of nilotinib or pharmaceutically acceptable salts thereof. In another embodiment, the suspension comprises about 30% w/v of nilotinib or pharmaceutically acceptable salts thereof.

In one aspect of any of the aqueous oral suspension described herein, are the one or more acid. Suitable acids include but are not limited to acetic acid, ascorbic acid, citric acid, fumaric acid, lauric acid, glacial, sorbic acid, malic acid, succinic acid, tartaric acid, phosphoric acid, hydrochloric acid, amino acid and mixtures thereof. Organic acids, which enhance the taste of the suspension, are especially useful. Citric acid, for example, adds tartness that is a taste enhancer, and may play a role in overall stability of the suspension. Suitable amino acid include but are not limited to lysine, alanine, glutamic acid, tryptophan and mixture thereof. The concentration of the acid in the solvent system is in the range of about 0.1% to about 10.0% or about 0.25% to about 5.0% or about 0.5% to about 3% or about 2%.

In one aspect of any of the pharmaceutical suspension described herein, the one or more preservatives comprise Bronopol, Imidurea, Potassium Sorbate, Phenoxyethanol, Phenylmercuric Acetate, Butylparaben, Benzyl Alcohol, Phenylmercuric Borate, Chlorocresol, Benzethonium Chloride, Phenylethyl Alcohol, Benzalkonium Chloride, Methylparaben, Hexetidine, Chlorobutanol, Ethylparaben, Propylparaben, Sodium Benzoate, Potassium Benzoate, Sorbic Acid, Cresol, Propylparaben Sodium, Cetylpyridinium Chloride, Phenylmercuric Nitrate, Chloroxylenol, Propionic Acid, Phenol, Thimerosal, Sulfur Dioxide, Boric Acid, Edetic Acid, Sodium Propionate, Calcium Chloride, Sodium Acetate, Sodium Sulfite, Benzoic Acid, Monothioglycerol, Cetrimide, Calcium Acetate, Butylene Glycol, Sodium Metabisulfite, Alcohol, Propyl Gallate, Potassium Metabisulfite, Sodium Lactate, Chlorhexidine, Calcium Lactate, Pentetic Acid, Glycerin, Propylene Glycol Alginate, Sodium Borate, Magnesium Trisilicate, Isopropyl Alcohol, Dimethyl Ether, Propylene Glycol, Butylated Hydroxyanisole, Pyrrolidone, Lactic Acid, Sodium Lauryl Sulfate and Dimethyl Sulfoxide or a combination thereof. In another embodiment of any of the aqueous oral suspension described herein, the one or more preservatives comprise methylparaben, propylparaben, or a combination thereof. For instance, the oral suspension may comprise methylparaben and propylparaben.

For instance, the oral pharmaceutical suspension may comprise methylparaben and propylparaben. The suspension includes an effective amount of preservatives (such as methylparaben, propylparaben, sodium benzoate, sodium methylparaben, or a combination thereof) to inhibit microbial contamination of the suspension. In one embodiment, the suspension includes from about 0 to about 4% (such as from about 0 to about 1% or from about 0 to about 0.5%) of preservatives, such as methylparaben, propylparaben, or a combination thereof. In another embodiment, the suspension includes from about 0 to about 0.2% of preservatives, such as methylparaben, propylparaben, or a combination thereof.

In one aspect of any of the aqueous pharmaceutical suspension described herein, the suspension comprises one or more flavoring agents, such as, e.g., natural grape flavor. Other suitable flavoring agents include but, are not limited to, cotton candy, raspberry, strawberry, lemon-lime, apple, mint, licorice, orange, lemon, grapefruit, caramel, vanilla, cherry, grape flavors, tutti-frutti, cherry flavors, combinations thereof; and the like. In one embodiment, the suspension comprises from about 0.01 to about 2% w/v flavoring agent (e.g., natural grape flavor), such as about 1.0% w/v flavoring agent.

In some aspects, the stable oral pharmaceutical suspension of the present application comprises of at least one surfactant selected from the group of nonionic, anionic, cationic and zwitterionic surfactants or mixtures thereof.

Suitable non-limiting examples of the surfactants used in the compositions of the present application includes, but not limited to, polyethoxylated fatty acids like esters of lauric acid, oleic acid, and stearic acid, PEG-fatty acid diesters like PEG-20 dilaurate, PEG-fatty acid mono- and di-ester mixtures, alcohol-oil transesterification products like PEG-35 castor oil, Polyoxy 35 castor oil, polyoxyl 40 hydrogenated castor oil, etc., polyglycerized fatty acids like polyglyceryl oleate, etc., propylene glycol fatty acid esters like propylene glycol monolaurate etc., mixtures of propylene glycol esters-glycerol esters like oleic acid esters of propylene glycol and glycerol, etc., sterol and sterol derivatives like PEG-24 cholesterol ether etc., polyethylene glycol sorbitan fatty acid esters like PEG-20 sorbitan monolaurate etc., polyethylene glycol alkyl ethers like PEG-3 oleyl ether, etc., sugar esters like sucrose monopalmitate etc., polyethylene glycol alkyl phenols like Octoxynol-1 etc., sorbitan fatty acid esters like sorbitan monolaurate, lower alcohol fatty acid esters like ethyl oleate, etc., anionic surfactants include fatty acid salts and bile salts. Additional exemplary surfactants include, but are not limited to: polyoxyethylene alkylethers; polyethylene glycol fatty acid esters; polyethylene glycol glycerol fatty acid esters; polyoxyethylene sorbitan fatty acid esters; polyglycerol fatty acid esters; polyoxyethylene glycerides; polyoxyethylene vegetable oils; polyoxyethylene hydrogenated vegetable oils, alkylglucosides; alkylmaltosides; alkylthioglucosides; lauryl macrogolglycerides Ionic surfactants include sodium oleate, sodium lauryl sulfate, sodium lauryl sarcosinate, sodium dioctyl sulfosuccinate, sodium cholate, and sodium taurocholate; Gelucire® 44/14, etc.

The at least one surfactant be present in the stable oral liquid pharmaceutical suspension disclosed herein in a variety of concentrations. For example, the stable oral liquid pharmaceutical suspension disclosed herein can comprise at least one surfactant of at least 0.25% by weight, at least 2% by weight, at least 5% by weight, at least 10% by weight, at least 15% by weight, at least 30% by weight, or a percentage between any two of the above values, based on the total weight of the composition. In some embodiments, the nilotinib composition of present application comprises of at least one surfactant in an amount of from about 0.25% to about 20% by weight, or from about 2% to about 15% by weight, or from about 2% to about 10% by weight, or from about 3% to about 10% by weight, based on the total weight of the composition.

In one aspect of any of the aqueous oral suspension described herein, the pH of the aqueous oral suspension ranges from about 2 to about 7. In another of any of the aqueous oral suspension described herein, the pH of the aqueous oral suspension ranges from about 2.0 to about 4. For instance, the pH may be 2.7, 2.8, 2.9, 3, 3.1, 3.2, 3.3, 3.4, or 3.5.

In some aspects, the stable oral liquid pharmaceutical suspension of the present application may comprises of at least one buffering agents. Non-limiting examples of buffering agents include, but are not limited to acetate, amino acids, ammonium sulfate, benzoate, bicarbonate, borate, citrate, citric acid monohydrate, disodium hydrogen phosphate, glutamate, lactate, meglumine, potassium citrate, sodium acetate, sodium citrate, sodium phosphate, sulfate, tartrate, triethanolamine, TRIS, trisodium citrate dehydrate, and any combination thereof.

In one aspect of any of the aqueous oral suspension described herein, the suspension may comprise stabilizing agents. Suitable stabilizing agents which may be incorporated into composition of the present invention include, e.g., monovalent or multivalent metal cations. Non-limiting examples salts are inorganic salts, include calcium sulfate, sodium chloride, potassium sulfate, sodium carbonate, lithium chloride, tripotassium phosphate, sodium borate, potassium bromide, potassium fluoride, sodium bicarbonate, calcium chloride, magnesium chloride, sodium citrate, sodium acetate, calcium lactate, magnesium sulfate and sodium fluoride or any of the commonly employed salts.

In another aspect stabilizing agent may comprise multivalent metal cations. In another embodiments, the stabilizing agents are bivalent. In yet another embodiments, bivalent cation salts is sodium chloride.

In one aspect of any of the aqueous oral suspension described herein, the suspension comprises from about 0.1 to about 10% w/v of stabilizing agent, such as from about 0.2 to about 5% w/v of stabilizing agent.

In one aspect of any of the aqueous oral suspension described herein, the suspension comprises from about 0.1 to about 10% w/v of viscosity modifier, such as from about 0.2 to about 5% w/v of viscosity modifier.

Ionic salt and viscosity modifier mean the same for the purpose of this invention.

In one aspect of any of the aqueous oral suspension described herein, the suspension may comprises anti-oxidant. Non-limiting examples of anti-oxidant included, but are not limited to propyl gallate, lecithin, Vitamin E tocopherol, sesamin, sesamol, sesamolin, alpha tocopherol, ascorbic acid, ascorbyl palmitate, fumaric acid, citric acid, malic acid, and sodium metabisulphite, disodium EDTA, and combinations of any of the foregoing.

Another aspect of the present invention is to provide an aqueous oral suspension composition containing, while having a reduced bitter taste of the drug.

A further aspect of the present invention is to provide a suspension composition which is easy to take.

“Nilotinib” as used herein encompasses base form as well as its pharmaceutically acceptable salts, complexes, polymorphs, hydrates, solvates, enantiomers or racemates. The solid state form of nilotinib used in the composition of the present application is not critical. For example, nilotinib can be amorphous or crystalline.

The term “pharmaceutically acceptable salts” as used herein includes those salts which are, within the scope of sound medical judgment, suitable for use in contact with the tissues of humans and lower animals without undue toxicity, irritation, allergic response and the like, which are well known in the art. The salts can be prepared in situ during the final isolation and purification of the compounds of the invention, or separately by reacting the pharmaceutically active substance, having a freebase function, with a suitable organic acid or inorganic acid. Suitable examples include tartaric acid, Hydrobromide, Succinate, Glutamate, Acetate, L malate, L maleate, HCl, Fumarate, butanedisulfonic acid, gentisate, mono oxalate, di nitrate, Ethanedi sulfonic acid, Isethionic acid and Naphth alene-2-sulfonic acid etc.

As used herein, an “effective amount” or a “therapeutically effective amount” of a drug refers to a non-toxic, but sufficient amount of the drug, to achieve therapeutic results in treating a condition for which the drug is known to be effective. In this instance, an effective amount is an amount of nilotinib which is sufficient to treat leukemia in a patient in need thereof. The term “nilotinib” or a pharmaceutically acceptable salt thereof refers to nilotinib free base or any pharmaceutically acceptable salt of nilotinib, the pharmaceutical composition of the present invention may include nilotinib in amounts ranging from about 0.05% w/v to about 20% w/v of the composition.

The term “liquid suspension” refers to a liquid composition that is ingested with or without further mixing with aqueous or suitable media before oral administration.

Unless otherwise stated all concentrations mentioned herein are based on total weight of the composition.

As used herein, the term “flavoring agent” is intended to mean a compound used to impart a pleasant flavor and often odor to a pharmaceutical preparation. Exemplary flavoring agents include synthetic flavor oils and flavoring aromatics and/or natural oils, extracts from plants, leaves, flowers, fruits and so forth and combinations thereof. These may also include cinnamon oil, oil of wintergreen, peppermint oils, clove oil, bay oil, anise oil, eucalyptus, thyme oil, cedar leave oil, oil of nutmeg, oil of sage, oil of bitter almonds and cassia oil. Other useful flavoring agents include vanilla, citrus oil, including lemon, orange, grape (e.g., natural grape), lime and grapefruit, and fruit essences, including apple, pear, peach, strawberry, raspberry, cherry, plum, pineapple, apricot and so forth. The amount of flavoring agent may depend on a number of factors, including the organoleptic effect desired.

The inventive composition also comprises a sweetener. Various sweeteners are contemplated, including but not limited to simple sugars such as sucrose, dextrose, fructose, maltose, and the like. In other embodiments, the inventive composition is “low calorie” or “light”, “sugar-free”, or “calorie-free.” As discussed above, the sweetener in the inventive compositions may be a so-called “artificial sweetener” (also known as “high-intensity sweetener”), such as sucralose, acesulfame potassium, saccharin, and aspartame, or any combination thereof. The use of such artificial sweeteners is desirable for adding sweetness without the addition of calories. Also as discussed above, the polyol in the inventive composition may also provide some sweetening and the lower calorie content of polyols, and lower glycemic index (compared to simple sugars) make the inventive compositions suitable for low calorie diets. Additionally, the inventive compositions that are low calorie and/or low glycemic index would be suitable for diabetic patients.

One of skill in the art will recognize that the “sugar-free” means that a product contains no amount of, or only trivial or “physiologically inconsequential” amounts of sugars. In this regard, “sugar free” means less than 0.5 g of sugars per serving. “Calorie free” means fewer than 5 calories per serving. Examples of synonyms for “free” include “without,” “no” and “zero.” Those products sweetened only with artificial sweeteners and/or sugar alcohols (and containing no other sugars) can be classified as “sugar-free.” The term “low calorie” is understood to mean 40 calories or less per reference amount.

Other excipients conventionally used in pharmaceutical compositions can be included, and these excipients are well known in the art. Such excipients include pharmaceutically acceptable detackifiers, anti-foaming agents, chelating agents, viscomodulators, tonicifiers, flavorants, colorants odorants, opacifiers, suspending agents, binders, fillers, plasticizers, lubricants, and mixtures thereof. The amounts of such excipients can be readily determined by one skilled in the art, according to the particular properties desired, keeping in mind the possibility that any such excipients should preferably not negatively impact the stability of the final formulation.

Suitable coloring agents include red, black and yellow iron oxides and FD&C dyes such as FD&C Blue No. 2, FD&C Red No. 40, and the like. Suitable taste-masking agents include sodium bicarbonate, ion-exchange resins, cyclodextrin inclusion compounds, adsorbates, and the like.

It is recognized that pharmaceutical excipients may perform more than one function, and are therefore characterized as having different uses depending on the particular application. While the use of an excipient in the context of a particular formulation may determine the function of the excipient, the inclusion of any particular excipient into any one or more category as set forth above is not meant to limit the function of that excipient.

The following examples are provided to enable one of ordinary skill in the art to prepare dosage forms of the invention and should not be construed as limiting the scope of the invention.

### EXAMPLES

TABLE 1
Selection of surfactants

| Surfactants name | Concentration | | Solubility |
|---|---|---|---|
| Polysorbate 80 | 126 | mg/mL | 2.286 mg/mL |
| Polysorbate 20 | 5 | mg/ml | 0.478 mg/mL |
| Span 80 | 150 | ml/ml | Not done (phase separation observed |
| | | | and not miscible with API) |
| Poloxamer 188 | 100 | mg/ml | 0.321 mg/mL |
| Poloxomer 407 | 10 | mg/ml | 0.227 mg/mL |
| SLS | 5 | mg/ml | Not done (Large lump formed |
| | | | immedicably after addition API) |

Without surfactant API solubility was 0.2 μg/ml, and based on the above solubility study with different surfactant polysorbate 80 shows better solubility 2.286 mg/ml. Hence polysorbate 80 was selected for preparation of suspension.

TABLE 2
Formulas with different concentration of polysorbate 80

| | E1 | E2 | E3 | E4 | E5 | E6 |
|---|---|---|---|---|---|---|
| Raw Materials | (mg/ml) | (mg/ml) | (mg/ml) | (mg/ml) | (mg/ml) | (mg/ml) |
| Nilotinib or | 22 (20 | 22 (20 | 22 (20 | 22 (20 | 22 (20 | 22 (20 |
| pharmaceutically acceptable | mg/ml as | mg/ml as | mg/ml as | mg/ml as | mg/ml as | mg/ml as |
| salts thereof | Nilotinib) | Nilotinib) | Nilotinib) | Nilotinib) | Nilotinib) | Nilotinib) |
| Microcrystalline cellulose & | 30 | 30 | 30 | 30 | 30 | 30 |
| sodium carboxymethylcellulose | | | | | | |
| Xanthan Gum | 0.6 | 0.6 | 0.6 | 0.6 | 0.6 | 0.6 |
| Polyethylene Glycol (PEG400) | 240 | 240 | 240 | 240 | 240 | 240 |
| Polysorbate 80 | 5 | 3.5 | 2 | 1 | 0.5 | 0 |
| Simethicone Emulsion (30%) | 2 | 2 | 2 | 2 | 2 | 2 |
| Citric Acid anhydrous | 15.8 | 15.8 | 15.8 | 15.8 | 15.8 | 15.8 |
| Sodium Citrate Anhydrous | 3 | 3 | 3 | 3 | 3 | 3 |
| Propylparaben | 0.1 | 0.1 | 0.1 | 0.1 | 0.1 | 0.1 |
| Methylparaben | 1 | 1 | 1 | 1 | 1 | 1 |
| Sucrose | 300 | 300 | 300 | 300 | 300 | 300 |
| Sucralose | 2.4 | 2.4 | 2.4 | 2.4 | 2.4 | 2.4 |
| Strawberry Liquid | 0.1 | 0.1 | 0.1 | 0.1 | 0.1 | 0.1 |
| Sodium Chloride | 5 | 5 | 5 | 5 | 5 | 5 |
| Purified Water | q. s to | q. s to | q. s to | q. s to | q. s to | q. s to |
| | 1 mL | 1 mL | 1 mL | 1 mL | 1 mL | 1 mL |
| pH | 3.06 | 3.05 | 3.09 | 3.18 | 3.13 | 3.17 |

| | | |
|---|---|---|
| Observations | Satisfactory | Rapid |
| (Sedimentation study) | | sedimentation |
| | | observed |

TABLE 3
Sedimentation Data

| | |
|---|---|
| Time | Volume of Clear Liquid (mL) |

| (Hours) | E1 | E2 | E3 | E4 | E5 | E6 |
|---|---|---|---|---|---|---|
| 24 | 3 | 2 | 2 | 3 | 2 | 9 |
| 48 | 4 | 3 | 5 | 6 | 5 | 18 |
| 72 | 6 | 4 | 9 | 10 | 9 | 25 |

In Examples E1 to E6, polysorbate 80 in various concentrations was evaluated. The absence of polysorbate 80 lead to rapid sedimentation.

TABLE 4
Formulas with different concentration of NaCl

| | E1 | E7 | E8 | E9 | E10 | E11 |
|---|---|---|---|---|---|---|
| Raw Materials | (mg/ml) | (mg/ml) | (mg/ml) | (mg/ml) | (mg/ml) | (mg/ml) |
| Nilotinib Hydrochloride | 22 (20 | 22 (20 | 22 (20 | 22 (20 | 22 (20 | 22 (20 |
| Monohydrate | mg/ml as | mg/ml as | mg/ml as | mg/ml as | mg/ml as | mg/ml as |
| | Nilotinib) | Nilotinib) | Nilotinib) | Nilotinib) | Nilotinib) | Nilotinib) |
| Microcrystalline cellulose & | 30 | 30 | 30 | 30 | 30 | 30 |
| sodium carboxymethylcellulose | | | | | | |
| Xanthan Gum | 0.6 | 0.6 | 0.6 | 0.6 | 0.6 | 0.6 |
| Polyethylene Glycol (PEG400) | 240 | 240 | 240 | 240 | 240 | 240 |
| Polysorbate 80 | 5 | 5 | 5 | 5 | 5 | 5 |
| Simethicone Emulsion (30%) | 2 | 2 | 2 | 2 | 2 | 2 |
| Citric Acid anhydrous | 15.8 | 15.8 | 15.8 | 15.8 | 15.8 | 15.8 |
| Sodium Citrate Anhydrous | 3 | 3 | 3 | 3 | 3 | 3 |
| Propylparaben | 0.1 | 0.1 | 0.1 | 0.1 | 0.1 | 0.1 |
| Methylparaben | 1 | 1 | 1 | 1 | 1 | 1 |
| Sucrose | 300 | 300 | 300 | 300 | 300 | 300 |
| Sucralose | 2.4 | 2.4 | 2.4 | 2.4 | 2.4 | 2.4 |
| Strawberry Liquid | 0.1 | 0.1 | 0.1 | 0.1 | 0.1 | 0.1 |
| Sodium Chloride | 5 | 4 | 2.5 | 1 | 0.5 | 0 |
| Purified Water | q. s to | q. s to | q. s to | q. s to | q. s to | q. s to |
| | 1 mL | 1 mL | 1 mL | 1 mL | 1 mL | 1 mL |
| pH | 3.06 | 3.05 | 3.06 | 3.19 | 3.21 | 3.32 |

| Observations | Satisfactory | Initially ok; lump | Not |
|---|---|---|---|
| (Sedimentation study) | | formation observed | satisfactory; |
| | | after 7 days | yellowish |

in color;
lump formation
observed

Examples E1 & E7 to E11, Sodium chloride in various quantities is studied. As the quantity of NaCl decreases the sedimentation increases.

TABLE 5
Comparison of E1, E12 & E13

| | E1 | E12 | E13 |
|---|---|---|---|
| Raw Materials | (mg/ml) | (mg/ml) | (mg/ml) |
| Nilotinib Hydrochloride | 22 (20 mg/ml | 22 (20 mg/ml | 22 (20 mg/ml |
| Monohydrate | as Nilotinib) | as Nilotinib) | as Nilotinib) |
| Microcrystalline cellulose & | 30 | 25 | 20 |
| sodium carboxymethylcellulose | | | |
| Xanthan Gum | 0.6 | 0.5 | 0.4 |
| Polyethylene Glycol (PEG400) | 240 | 240 | 240 |
| Polysorbate 80 | 5 | 5 | 5 |
| Simethicone Emulsion (30%) | 2 | 2 | 2 |
| Citric Acid anhydrous | 15.8 | 15.8 | 15.8 |
| Sodium Citrate Anhydrous | 3 | 3 | 3 |
| Propylparaben | 0.1 | 0.1 | 0.1 |
| Methylparaben | 1 | 1 | 1 |
| Sucrose | 300 | 300 | 300 |
| Sucralose | 2.4 | 2.4 | 2.4 |
| Strawberry Liquid | 0.1 | 0.1 | 0.1 |
| Sodium Chloride | 5 | 5 | 5 |
| Purified Water | q.s to 1 mL | q.s to 1 mL | q.s to 1 mL |
| pH | 3.06 | 3.02 | 3.06 |

| | |
|---|---|
| Observations | Satisfactory |

TABLE 6
Sedimentation Data

| Time | Volume of Clear Liquid (mL) | | |
|---|---|---|---|
| (Hours) | E1 | E12 | E13 |
| 24 | 3 | 7 | 5 |
| 48 | 4 | 14 | 9 |
| 72 | 6 | 18 | 13 |

| | |
|---|---|
| Observation | Satisfactory |

TABLE 7
Process for preparing the formulations

| Steps | Process |
|---|---|
| 1 | Add sucrose into purified water and heat up to 80° C. and mix until completely dissolved. |
| | Cool down to room temperature. Then add NaCl and sucralose in sucrose solution. |
| 2 | Add Microcrystalline cellulose & sodium carboxymethylcellulose into step-1 and mix for 30 |
| | minutes using silverson homogenizer at 2500-3000 RPM |
| 3 | Take purified water in a breaker and add citric acid and mix until dissolved |
| 4 | Take purified water in a breaker and add Xanthan Gum and mix for 20-30 minutes at 350 |
| | RPM |
| 5 | Add methylparaben and propylparaben into PEG 400 and mix for 30 minutes at 350 RPM |
| | with stirrer |
| | Add Strawberry Flavor, Simethicone emulsion 30% and Tween 80 and mix for 20 minutes |
| | using silverson homogenizer at 1200-1500 RPM |
| 6 | Add step 3 into step 5 |
| 7 | Add API into step 6 and mix for 30 minutes using silverson homogenizer at 1200-1500 RPM |
| 8 | Add step 2 into step 7 and mix for 20 minutes using silverson homogenizer at 1200- |
| | 1500 RPM |
| 9 | Add step 4 into step 8 and mix for 20 minutes using silverson homogenizer at 1200- |
| | 1500 RPM |
| 10 | Take purified water in a beaker and add sodium citrate anhydrous and mix until dissolved |
| | and add to step 9 and mix for 10 minutes using silverson homogenizer at 1200-1500 RPM |
| 11 | Adjust the volume and final mixing for 45-60 minutes at 1200-1500 RPM |

TABLE 8
Stability data of E1

| | Batch No.: | | | |
|---|---|---|---|---|
| | E1 | | | |
| | | | 40° C./75% | |
| Parameters | Specification | Initial | 3 months | 6 months |
| Assay | 90.0% to 110.0% of label | 102.9 | 101.0 | 99.0 |
| | amount of Nilotinib | | | |
| pH | Between 2.50 & 3.50 | 3.00 | 2.98 | 2.94 |
| Density (Wt/mL) | Between 1.05 g/mL & | 1.175 | 1.176 | 1.176 |
| | 1.25 g/mL | | | |
| Viscosity | 60-100 cps at 25° C. 15 RPM | 75.20 | 70.80 | 70.00 |
| | with spindle SC4-18 | | | |
| Redispersibility | Easily redispersible after | Easily | Easily | Easily |
| | 30 seconds shaking | Redispersible | redispersible | redispersible |
| Related | Impurity-A: NMT 0.2% | ND | ND | ND |
| Substances | Impurity-B: NMT 0.2% | ND | ND | ND |
| | Impurity-C: NMT 0.2% | ND | ND | ND |
| | Impurity-D: NMT 0.2% | ND | ND | ND |
| | Impurity-E: NMT 0.2% | ND | ND | ND |
| | Impurity-F: NMT 0.2% | ND | 0.0336 | 0.0506 |
| | Impurity-G: NMT 0.2% | ND | ND | ND |
| | Impurity-H: NMT 0.2% | ND | ND | ND |
| | Highest unknown impurity: | ND | 0.0585 | 0.1134 |
| | NMT 0.2% | | | |
| | Total Impurity NMT 2% | 0.00 | 0.0921 | 0.164 |
| Microbial | TAMC: NMT 100 cfu/ml | <10 cfu/ml | Not Done | <10 cfu/ml |
| Limit Test | TYMC: NMT 10 cfu/ml | <10 cfu/ml | | 05 cfu/ml |
| | E. Coli: Absent/ml | Absent/ml | | Absent/ml |

ND: Not detected

TABLE 9
Dissolution in 0.1N HCl, Apparatus-II, RPM-50, Volume 900 mL

Batch Number: (E1)

| | | 40° C./75% |
|---|---|---|
| Time (Mins) | Initial | 3 months |
| 5 | 97 (95-100); 1.85% | 101 (100-101); 0.54% |
| 10 | 100 (99-101); 0.63% | 100 (99-101); 0.75% |
| 15 | 101 (101-102); 0.51% | 100 (99-101); 0.82% |
| 30 | 102 00-103); 1.08% | 101 (99-102); 1.04% |
| 45 | 102 (101-103); 0.87% | 101 (100-101); 0.54% |

### Dissolution Method:

Apparatus-II (Paddle)
RPM-50,
Volume: 900 mL
Media: 0.1 N HCL
Temperature: 37° C.
Time point: 5 min, 10 min, 15 min, 30 min and 45 minutes

TABLE 10
Release media Dissolution data
Dissolution in 0.1N HCl, Apparatus-II, RPM-50, Volume 900 mL

| | Tasigna 200 | Batch Numbers |
|---|---|---|
| Time (Mins) | mg capsule* | (E1) |
| 5 | 98 (96-99); 1.07% | 97 (95-100); 1.85% |
| 10 | 98 (97-100); 1.29% | 100 (99-101); 0.63% |
| 15 | 98 (97-100); 1.23% | 101 (101-102); 0.51% |
| 30 | 98 (97-100); 1.19% | 102 (100-103); 1.08% |
| 45 | 98 (97-100); 1.05% | 102 (101-103); 0.87% |
| Infinity | 99 (97-100); 1.06% | 102 (101-103); 0.74% |

*Capsule content in one teaspoon of applesauce

Selection of 0.1N HCl as release media:

| Media | API Solubility (mg/mL) |
|---|---|
| Water | 0.081 |
| 0.1N HCl | 1.982 |
| 0.01N HCl | 0.494 |
| 0.001N HCl | 0.126 |
| pH 4.5 acetate buffer | 0 |
| pH 6.8 phosphate buffer | 0 |

0.1N HCl is selected as release media as it shows highest solubility of API

TABLE 11
Multimedia Dissolution data
Table 11.1 Dissolution in pH 4.5 Acetate buffer +
0.5% SLS, Apparatus-II, RPM-50, Volume 900 mL

| | Tasigna 200 | Batch Numbers |
|---|---|---|
| Time (Mins) | mg capsule* | (E1) |
| 5 | 70 (69-71); 1.17% | 37 (33-42); 8.95% |
| 10 | 76 (75-77); 0.99% | 48 (46-53); 5.69% |
| 15 | 80 (78-82); 1.71% | 58 (54-62); 4.71% |
| 30 | 85 (83-88); 2.11% | 77 (75-81); 2.65% |
| 45 | 88 (84-91); 3.27% | 86 (82-91); 3.65% |
| Infinity | 87 (85-90); 2.01% | 92 (90-94); 1.64% |

*Capsule content in one teaspoon of applesauce

TABLE 11.2
Dissolution in pH 6.8 Phosphate Buffer +
0.15% SLS, Apparatus-II, RPM-50, volume 900 mL

| | Tasigna 200 | Batch No |
|---|---|---|
| Time (Mins) | mg capsule* | E1 |
| 5 | 20 (19-21); 4.10% | 30(26-34); 9.53% |
| 10 | 25 (24-26); 3.00% | 38(34-41); 7.00% |
| 15 | 29 (29-30); 1.41% | 43(40-46); 5.63 |
| 30 | 39 (38-40); 1.62% | 55(53-57); 2.67% |
| 45 | 46 (44-47); 2.54% | 64(62-66); 2.30% |
| Infinity | 49 (45-52); 5.98% | 67(65-69); 2.19% |

*Capsule content in one teaspoon of applesauce

TABLE 13
Comparison of dissolution with graph with various excipient.
Dissolution in pH 4.5 Acetate buffer + 0.5% SLS, Apparatus-II, RPM-50, Volume 900 mL

| | Tasigna 200 | Batch Numbers | | | |
|---|---|---|---|---|---|
| Time (Mins) | mg capsule* | (E5) | (E4) | (E3) | (E1) |
| Polysorbate 80 | NA | 0.5 | 1.0 | 2.0 | 5.0 |
| (mg/mL) | | | | | |
| 5 | 70 (69-71); 1.17% | 52 (44-57); 9.10% | 45 (32-64); 30.27% | 31 (28-35); 8.00% | 80 (72-90); 7.93% |
| 10 | 76 (75-77); 0.99% | 59 (53-65); 7.47% | 59 (44-72); 19.10% | 47 (44-52); 5.77% | 88 (82-95); 5.66% |
| 15 | 80 (78-82); 1.71% | 63 (59-69); 5.59% | 68 (56-79); 12.91% | 59 (57-63); 3.56% | 92 (88-95); 3.13 |
| 30 | 85 (83-88); 2.11% | 75 (72-81); 4.41% | 91 (87-94); 3.16% | 85 (84-87); 1.21% | 99 (96-101); 1.85% |
| 45 | 88 (84-91); 3.27% | 81 (77-87); 4.62% | 96 (93-97); 1.53% | 96 (95-97); 0.85% | 100 (97-101); 1.76% |
| Infinity | 87 (85-90); 2.01% | 89 (83-93); 4.79% | 97 (95-98); 1.21% | 97 (96-98); 0.65% | 99 (96-101); 2.16% |

*Capsule content in one teaspoon of applesauce

TABLE 14
Dissolution in pH 6.8 Phosphate Buffer + 0.15% SLS, Apparatus-II, RPM-50, volume 900 mL

| | Tasigna 200 | Batch Numbers | | | |
|---|---|---|---|---|---|
| Time (Mins) | mg capsule* | E5 | E4 | (E3) | (E1) |
| Polysorbate 80 | NA | 0.5 | 1.0 | 2.0 | 5.0 |
| (mg/mL) | | | | | |
| 5 | 20 (19-21); 4.10% | 16 (14-17): 7.31% | 16 (14-20); 13.94% | 20 (18-22); 8.20% | 23 (20-31); 17.61% |
| 10 | 25 (24-26); 3.00% | 26 (24-27); 4.50% | 25 (23-26); 4.88% | 27 (25-29); 6.89% | 34 (31-40); 9.62% |
| 15 | 29 (29-30); 1.41% | 34 (32-35); 3.03% | 31 (29-32); 3.39% | 32 (30-35); 6.75% | 81 (79-82); 1.30% |
| 30 | 39 (38-40); 1.62% | 51 (49-51); 1.65% | 44 (43-48); 4.41% | 45 (40-48); 6.24% | 86 (83-92); 3.80% |
| 45 | 46 (44-47); 2.54% | 62 (61-63); 1.02% | 51 (49-53); 3.04% | 53 (51-55); 3.51% | 96 (91-98); 3.00% |
| Infinity | 49 (45-52); 5.98% | 68 (67-68); 0.76% | 52 (52-53); 0.79% | 55 (55-56); 0.95% | 95 (94-96); 0.66% |

*Capsule content in one teaspoon of applesauce

TABLE 15
Dissolution in 0.1N HCl, Apparatus-II, RPM-50, Volume 900 mL

| | Tasigna 200 | Batch Numbers | |
|---|---|---|---|
| Time (Mins) | mg capsule* | (E5) | (E1) |
| Polysorbate 80 | NA | 0.5 | 5.0 |
| (mg/mL) | | | |
| 5 | 98 (96-99); 1.07% | 97(92-100); 3.23% | 100 (99-101); 0.75% |
| 10 | 98 (97-100); 1.29% | 99(96-101); 2.16% | 100 (99-100); 0.55% |
| 15 | 98 (97-100); 1.23% | 99(97-101); 1.65% | 99 (99-100); 0.53% |
| 30 | 98 (97-100); 1.19% | 100(99-101); 0.98% | 99 (98-100); 0.64% |
| 45 | 98 (97-100); 1.05% | 100(98-101); 1.21% | 99 (98-99); 0.56% |
| Infinity | 99 (97-100); 1.06% | 101(100-102); 0.83% | 99 (98-99); 0.56% |

*Capsule content in one teaspoon of applesauce

1. A pharmaceutical oral suspension comprising:
Nilotinib or a pharmaceutically acceptable salt thereof
Water;
a suspending agent;
a stabilizing agent;
a buffering agent and one or more pharmaceutically acceptable excipients

2. The pharmaceutical oral suspension of claim 1 wherein the suspending agent is selected from gelatin, crosslinked polyacrylic acid, polymethacrylic acid, polyhydroxyethyl methacrylic acid, hydroxypropyl methyl cellulose, polyethylene glycol, sodium carboxymethyl cellulose, hyaluronic acid, chitosan, polycarbophil, pectin, copolymers of dextran, polyacrylamide, acacia, copolymer of caprolactone and ethylene oxide, carbopol 934, tragacanth, eudragit. polyvinyl pyrrolidone, polyacrylate and polyacrylate copolymer resins, celluloses and cellulose derivatives for example methyl-, ethyl- and propyl celluloses; hydroxyalkyl-celluloses, hydroxyl propyl celluloses, hydroxylpropylalkyl celluloses and the like including xanthan gum, polyvinyl resins, polyethylene glycol, polyethylene oxide, sorbitol, sucrose, xylitol, dextrose, fructose, maltitol, sugar, sodium alginate, or a combination thereof.

3. The pharmaceutical oral suspension of claim 1 wherein the stabilizing agent is selected from calcium sulfate, sodium chloride, potassium sulfate, sodium carbonate, lithium chloride, tripotassium phosphate, sodium borate, potassium bromide, potassium fluoride, sodium bicarbonate, calcium chloride, magnesium chloride, sodium citrate, sodium acetate, calcium lactate, magnesium sulfate and sodium fluoride or any of the commonly employed salts.

4. The pharmaceutical oral suspension of claim 1 wherein the stabilizing agent is sodium chloride.

5. The pharmaceutical oral suspension of claim 1 having a pH of about 2 to about 4.

6. The pharmaceutical oral suspension of claim 1 wherein the nilotinib or pharmaceutically acceptable salt thereof is present at about 1 to about 50% w/v.

7. The pharmaceutical oral suspension of claim 1 wherein the stabilizing agent is present at about 0.1 to about 10% w/v.

8. The pharmaceutical oral suspension of claim 1 wherein the suspending agent is polyethylene glycol.

9. The pharmaceutical oral suspension of claim 1 wherein the buffering agent is selected from acetate, amino acids, ammonium sulfate, benzoate, bicarbonate, borate, citrate, citric acid monohydrate, disodium hydrogen phosphate, glutamate, lactate, meglumine, potassium citrate, sodium acetate, sodium citrate, sodium phosphate, sulfate, tartrate, triethanolamine, TRIS, trisodium citrate dehydrate, and any combination thereof.

10. The pharmaceutical oral suspension of claim 1 wherein the one or more excipient is solubiliser.

11. A pharmaceutical oral suspension comprising:
nilotinib or a pharmaceutically acceptable salt thereof;
Water;
a suspending agent;
an ionic salt and
one or more pharmaceutically acceptable excipients.

12. The pharmaceutical oral suspension of claim 11 wherein the suspending agent is selected from gelatin, crosslinked polyacrylic acid, polymethacrylic acid, polyhydroxyethyl methacrylic acid, hydroxypropyl methyl cellulose, polyethylene glycol, sodium carboxymethyl cellulose, hyaluronic acid, chitosan, polycarbophil, pectin, copolymers of dextran, polyacrylamide, acacia, copolymer of caprolactone and ethylene oxide, carbopol 934, tragacanth, eudragit. polyvinyl pyrrolidone, polyacrylate and polyacrylate copolymer resins, celluloses and cellulose derivatives for example methyl-, ethyl- and propyl celluloses; hydroxyalkyl-celluloses, hydroxyl propyl celluloses, hydroxylpropylalkyl celluloses and the like including xanthan gum, polyvinyl resins, polyethylene glycol, polyethylene oxide, sorbitol, sucrose, xylitol, dextrose, fructose, maltitol, sugar, sodium alginate, or a combination thereof.

13. The pharmaceutical oral suspension of claim 11 wherein the ionic salt is selected from calcium sulfate, sodium chloride, potassium sulfate, sodium carbonate, lithium chloride, tripotassium phosphate, sodium borate, potassium bromide, potassium fluoride, sodium bicarbonate, calcium chloride, magnesium chloride, sodium citrate, sodium acetate, calcium lactate, magnesium sulfate and sodium fluoride or any of the commonly employed salts.

14. The pharmaceutical oral suspension of claim 11 wherein the ionic salt is sodium chloride.

15. The pharmaceutical oral suspension of claim 11 having a pH of about 2 to about 4.

16. The pharmaceutical oral suspension of claim 11 wherein the nilotinib is present at about 1 to about 50% w/v.

17. The pharmaceutical oral suspension of claim 1 wherein the ionic salt is present at about 0.1 to about 10% w/v.

18. The pharmaceutical oral suspension of claim 11 wherein the suspending agent is polyethylene glycol.

19. The pharmaceutical oral suspension of claim 11 wherein the buffering agent is selected from acetate, amino acids, ammonium sulfate, benzoate, bicarbonate, borate, citrate, citric acid monohydrate, disodium hydrogen phosphate, glutamate, lactate, meglumine, potassium citrate, sodium acetate, sodium citrate, sodium phosphate, sulfate, tartrate, triethanolamine, TRIS, trisodium citrate dehydrate, and any combination thereof.

20. The pharmaceutical oral suspension of claim 11 wherein the one or more excipient is solubiliser.
